(19) United States
(12) Patent Application Publication
(10) Pub. No.: US 2021/0299898 A1
(43) Pub. Date: Sep. 30, 2021

(54) RAZOR WITH NATURAL HEAD-HANDLE ANGLE

(71) Applicant: Jessica McDaniel, (US)

(72) Inventors: Jessica McDaniel, Mojave, CA (US); Stephane Gobron, San Gabriel, CA (US)

(21) Appl. No.: 17/246,615

(22) Filed: May 1, 2021

Related U.S. Application Data
(60) Provisional application No. 63/003,134, filed on Mar. 31, 2020.

(51) Int. Cl. B26B 21/52; B26B 21/22

(52) U.S. Cl. CPC B26B 21/522; B26B 21/521; B26B 21/225; B26B 21/227

(57) ABSTRACT

The present invention discloses a shaving razor which comprises a razor head, a razor head cartridge having a plurality of razor blades mounted on a razor blade platform and a rear edge portion and a front edge portion facing opposite to each other and a razor handle having a curved profile which is positioned upwards, deviating away from razor body. The razor handle is oriented at any desired angle as required by a user relative to contact surface of the razor head, thereby enabling the user to grab the razor handle with fingers resting close to rear surface of the razor head. The handle has an overall curved profile that is oriented upward. The handle is oriented preferably at a 80° angle relative to the anatomy-contacting surface of the head, the range is 50° to 150°, preferably 60° to 105°, and more preferably 70° to 95°.

### TECHNICAL FIELD

Embodiments of the present invention relate to shaving razors. In particular, embodiments of the present invention relate to shaving razor used for shaving purposes with an aim of achieving a smoother shave. More particularly, embodiments of the present invention relate to the shaving razor which is adjustable in a manner that a better handing of the shaving razor is enabled to control the force applied by razor head to an area which needs to be shaved.

BACKGROUND ART

Over the years, the shaving razor model has evolved to mean any business practice in which a company offers its customers a one-time shaving razor blade model, usually at little or no cost. This one-time shaving razor blade model is usually single edged or double edged. The use of these shaving razor blade models is much more common among men. Before the advent of razors, there was a concept of using water and a sharp tool to remove the hair by pulling the hair out. Razors are commonly made from stainless steel, honed to a razor-sharp edge and coated with even harder materials such as diamond like carbon. The razors are routinely replaced after cutting materials far softer than the blades themselves. Many shaving razor systems have been developed to provide user smooth shaving on the area which they wish to shave. But there have been many issues and challenges which are associated with these modern-day shaving razor systems which have been described in detail below.

US Patent application no. “US10899030B2” titled “Razor handle with a pivoting portion” discloses a razor system having a main body and a pivoting head pivotally coupled with the main body about a pivot axis. The pivoting head includes a base member and a cover member that over lies the base member in a mating manner. The cover member includes a face defining at least one exterior opening and the pivoting head can have an interior compartment in fluid communication with the main body and the exterior opening. The pivoting head can rotate from a first position at zero degrees to a second position at about 45 degrees relative to the first position, and any position there between. The handle of this razor system is not able to orient at 90 degrees from the head, thereby unable to allow grip close to back surface of the pivoting head. Also, one of the major drawback in this razor system design is that it leads to shaving induced irritation and is inableto provide user a smoother shave if the orientation with respect to the razor head goes above 45°.

Another shaving razor system technology involves the use of a shaving razor that can include a razor handle configuration that reduces the propensity for the shaving razor to roll in a user's hand and improves the maneuverability of the razor cartridge during shaving. In addition, the shaving razor can include a biasing pivoting member producing a progressively increasing return torque on the razor cartridge that forces the cartridge into flat contact with the skin thus improving glide and shaving closeness. The cartridge includes a connecting member and a cartridge pivot axis providing an axis of rotation for the cartridge. But the major drawback in this razor system technology is that the shaving razor is that it fails to enable a better handling of the razor to control the force applied by the razor head to the anatomy to be shaved. Due to this, a lack of grip is there which is needed by the user on the anatomy to be shaved to get a smoother shave.

Disposable grooming razors are well known in the art. A typical razor sold today involves a multi-blade cartridge that is mounted on an elongate plastic handle, and the razor is used until the blades become dull. Some razors are completely discarded when the blades become dull, and some razors accept fresh blade cartridges to replace dull blades. The handle and head may be retained for further use after the cartridge is discarded. But again, the biggest challenge with these disposable grooming razors is that they lead to irritation while using the head of these razors on the anatomy which needs to be shaved. These commercially available disposable grooming razors are not designed to enable the user to grab the handle just behind the razor head which leads to inability of the razor to provide smoother shave to the user who uses it.

Thus, there is a need for development of an efficient, convenient, smooth and easy to use shaving razor for shaving purposes that minimizes shaving induced irritations, provides a grip needed by the user on the anatomy to be shaved to get smoother shave and enable the user to grab the handle just behind the razor head which leads to ease of using the shaving razor by the user.

Further, limitations and disadvantages of conventional and traditional approaches will become apparent to the one skilled in the art through comparison of the described system with some aspects of the present disclosure, as set forth in the remainder of the present application and with reference to the drawings.

### SUMMARY

The summary of certain aspects of the present invention provided below are disclosed by way of example, and are not intended to be limiting. From a consideration of the foregoing problems as already described, a shaving razor for shaving purposes is disclosed which helps in overcoming the foregoing problems in prior technologies.

The present invention provides an effective and reliable approach forproviding users a smoother shave by enabling the users to grab handle positioned normally behind razor head which leads to the shaving razor providing smoother shave to the user who uses it. The present invention provides the shaving razor with the aim of reducing shaving-induced irritation and achieving a smoother shave. The design of the razor enables a better handling of the razor to control the force applied by the razor head to the anatomy to be shaved. The handle of this razor is oriented at approximately 90° angle from front surface of the razor head and allow for a grip close to the back surface of the razor head.

According to an embodiment, the shaving razor comprises a razor head, a razor head cartridge having a plurality of razor blades mounted on a razor blade platform and a rear edge portion and a front edge portion facing opposite to each other and a razor handle having a curved profile which is positioned upwards, deviating away from razor body, wherein the razor handle is oriented at any desired angle as required by a user relative to contact surface of the razor head, thereby enabling the user to grab the razor handle with fingers resting close to rear surface of the razor head. The handle is oriented preferably at a 90° angle relative to anatomy-contacting surface of the head. The range of orientation is 50° to 150°, preferably 65° to 105°, more preferably 70° to 95°. This razor can have a very short handle which can have a 10-60 mm (millimeters) length, preferably 15-30 mm. The portion of the handle connected to the razor head is more or less straight having length of 10-30 mm. The handle can have a 80-180 mm conventional length, and is designed at the conventional length of 110 mm. The shaving razor of the present invention provides the user with just enough space to comfortably grab and manipulate the razor. The handle can be a rod-like component, have a more flattened design with a wider cross section, or any other suitable shape. An additional design feature is included to allow the razor to lay on a flat surface while maintaining the head above resting surface. The handle is connected to back of the razor head, optionally the design includes a pivot articulation with a spring back action to push the head toward the anatomy.

According to one embodiment, the razor handle is having conventional length of 80-180 mm, thereby providing the user enough space to change grip and direction of the shaving razor. The handle has an overall curved profile that is oriented upward that is, going away from the razor body.The contact surface of the razor head is flat or slightly convex with a large radius of curvature. The shaving razor comprising the rear edge portion and the front edge portion of the razor head can be slightly recessed from front face of the razor head. The razor head comprises a frame to protect the contact surface of the razor head from corners of the plurality of blades. The plurality of razor blades comprises spring mounted or support mounted blades which allows movement of the plurality of razor blades within the razor head. The shaving razor also comprises a pivot axis of the razor handle positioned towards bottom surface of the razor head and parallel to the cutting edges of the plurality of razor blades.

According to another embodiment, the handle is designed in a way to lay in a stable fashion when resting on a flat surface such as countertop or table, while maintaining the razor head away from the table. This can be achieved in many different fashions: a flat on the bottom portion of the handle, or two small resting surface/features away from each other, or any other suitable mean. Angle between the front surface of the shaving razor and the distal portion of handle is approximately 90°. The handle is curved in an “upward” fashion going away from the 0° direction defined by the shaving direction. The handle is curved, and has a 65°-95° orientation with respect to the razor head front surface. It enables grabbing the handle just beyond the back surface of the razor head.

These and other objects and advantages of the invention will become apparent from the following description of certain present preferred embodiments taken in conjunction with the appended claims and the attached drawings. Additional features and advantages of embodiments of the invention will be described hereinafter which form the subject of the claims of the invention. The conception and specific embodiment disclosed herein may be readily utilized as a basis for modifying or designing other structures for carrying out the same or similar purposes of the present invention. Please note that such equivalent constructions do not depart from the scope of the invention as set forth in the appended claims. The novel features which are characteristic of the invention, both as to its organization and method of operation, together with further objects and advantages, will be better understood from the following description when considered in connection with the accompanying figures. Each of the figures is provided for the purpose of illustration and description only and is not intended as a limitation of the scope of the present invention or appended claims.

The following detailed description which is to be described in further detail is illustrative and is not intended to be in any way limiting. In addition to the illustrative aspects, embodiments, and features described above, further aspects, embodiments, and features will be apparent by reference to the following detailed description in conjunction with the accompanying drawings.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates a perspective view of a shaving razor, in accordance with one embodiment of the present invention.

FIG. 1B illustrates a perspective view of an exemplary embodiment of the shaving razor, in accordance with another embodiment of the present invention;

FIG. 2A illustrates an angular reference between razor head and handle of the shaving razor, in accordance with first embodiment of the present invention;

FIG. 2B illustrates an angular range between the head and the handle of the shaving razor, in accordance with second embodiment of the present invention;

FIG. 2C illustrates placement of the holding fingers of the user to pinch the shaving razor, in accordance with an embodiment of the present invention;

FIG. 2D illustrates currently commercialy-available razor handle design;

FIG. 2E illustrates placement of fingers of the user to hold the shaving razor, in accordance with an embodiment of the present invention;

FIG. 3A-3E illustrates different types of handle designs with their respective profiles, in accordance with an embodiment of the present invention;

FIG. 4A illustrates a handle design type having its respective profile in accordance with one embodiment of the present invention;

FIG. 4B illustrates a handle design type having its respective profile in accordance with another embodiment of the present invention;

FIG. 5A illustrates various head-handle angle variations in accordance with an embodiment of the present invention;

FIG. 5B illustrates head-handle attachment point location variations in accordance with another embodiment of the present invention;

FIG. 6 illustrates an exemplary embodiment of the handle with increasing thickness in accordance with an embodiment of the present invention;

FIG. 6A illustrates an exemplary embodiment of a conventional-length handle with a preferred profile in accordance with another embodiment of the present invention;

FIG. 7 illustrates a perspective view of the handle of the shaving razor lying on a flat surface in accordance with an embodiment of the present invention;

FIG. 8 illustrates head-handle pivot articulation and spring action in accordance with an embodiment of the present invention;

FIG. 9 illustrates razor head, blade, cutting angle and recess in accordance with an embodiment of the present invention;

FIG. 9A illustrates angulation of blades in accordance with one embodiment of the present invention;

FIG. 9B illustrates angulation of blades in accordance with another embodiment of the present invention;

FIG. 10 illustrates a razor head with finite number of blades in accordance with an embodiment of the present invention; and

FIG. 10A illustrates variation in shape of razor heads having blade of different lengths in accordance with another embodiment of the present invention.

### DETAILED DESCRIPTION

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

In the following detailed description, various specific details are set forth in order to provide an understanding of and describe the devices and techniques introduced here. However, the techniques may be practiced without the specific details set forth in these examples. Various alternatives, modifications, and/or equivalents will be apparent to those skilled in the art without varying from the spirit of the introduced mechanical components and techniques. For example, while the embodiments described herein refer to particular features, the scope of this solution also includes embodiments having different combinations of features and embodiments that do not include all of the described features. Accordingly, the scope of the techniques and solutions introduced herein are intended to embrace all such alternatives, modifications, and variations as they fall within the scope of the claims, together with all equivalents thereof. Therefore, the description should not be taken as limiting the scope of the invention, which is defined by the claims.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

References to “one embodiment,” “alternate embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an embodiment”, “another embodiment,” do not necessarily refer to the same embodiment, although they may.

As it is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In various circumstances it can be advantageous for ashaving razor having the handle is designed to enable the user to grab the handle with the fingers resting very close to rear surface of the razor head, that is, within 0-30 mm from the rear surface of the razor head. The handle has an overall curved profile that is oriented upward that is, going away from the body. A design feature is included to allow the razor to lay on a flat surface while maintaining the head above the resting surface. The handle is oriented preferably at a 90° angle relative to the anatomy-contacting surface of the head, the range is 50° to 150°, preferably 60°-105°, more preferably 70°-95°. This razor can have a very short handle, that is, a conventional length of 80-180 mm, preferably 10-30 mm, to provide the user with just enough space to comfortably grab and manipulate the razor.

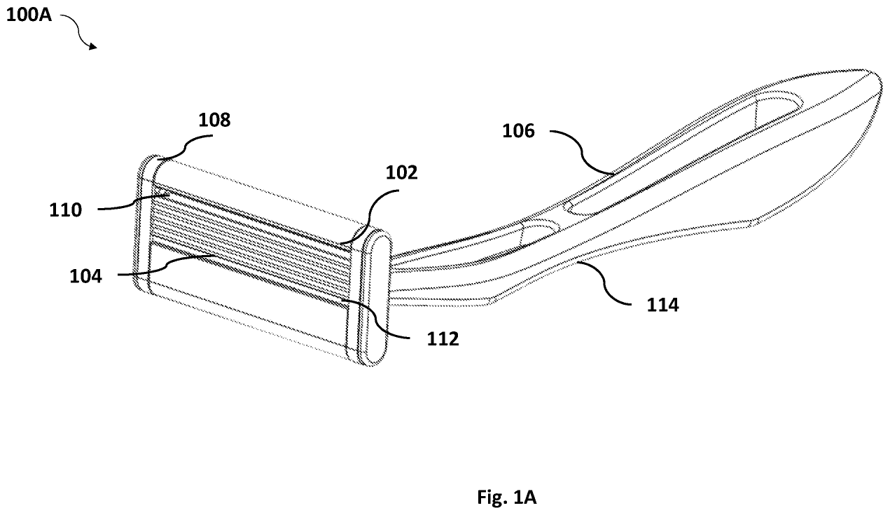

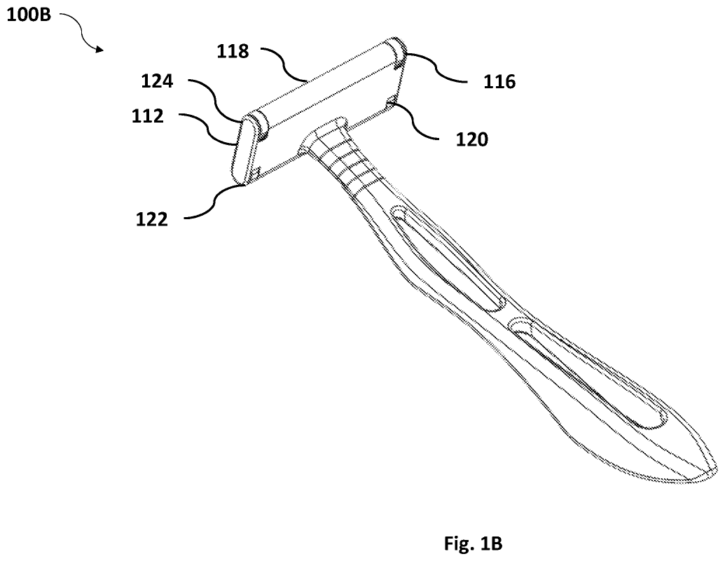

FIG. 1A illustrates a perspective view of the shaving razor 100A used for shaving purposes. The shaving razor 100A comprises a razor head 102, a plurality of razor blades 110 mounted on a razor blade platform 108 and a rear edge portion 126 and a front edge portion 112 facing opposite to each other and a razor handle 114 having a curved profile which is positioned upwards, deviating away from razor body 106. The shaving razor 100A may or may not comprise a razor head cartridge 104 as shown in the FIG. 1A. The razor handle 114 is oriented at approximately 90° angle (range 50°-150°, preferably 60°-105°, more preferably 70°-95°) from the front surface of the razor head 102, thereby enabling the user to grab the razor handle 114 with fingers resting close to rear surface of the razor head 102. The shaving razor 100A of the present invention is able to provide an improved closer shave to skin covered with hair by forcing the plurality of razor blades 110 of the razor head cartridge 104 into a more even contact with the skin. Front surface of the razor head 102 is placed against the anatomy to be shaved. The design of the shaving razor 100A of the present invention enables a better handling of the razor 100A to control the force applied by the razor head 102 to the anatomy to be shaved.

Optionally, a zone of attachment between the handle 114 and the head 102, can be setup along with a pivot articulation and its axis. The pivot axis is preferably located toward the bottom surface of the razor head 102 and its axis is parallel to the cutting edges of the plurality of razor blades 110. Optionally, a pushback force generating mechanism is part of the design of the shaving razor 100. Alternatively, the user's fingers would act to maintain the head 102 in position instead of an integrated push-back mechanism. The orientation between distal end of the razor handle and the front surface of the razor head can be adjusted at any desired angle as required by the user.

Referring to FIG. 1B illustrating a perspective view of an exemplary embodiment of the shaving razor 100B which includes a front edge 122 and a rear edge 124. The razor 100B as shown in FIG. 1B comprises a head 102 and a handle 114 as a single design. It is preferred that the head 102 be of a standard size, i.e., a size similar and/or substantially close to the dimensions of presently available razors. The handle 114 grips the razor head 102 and the handle 114 comprises a clip 120 for maintaining the individual blades into the razor head 102 or handle 114. The razor head cartridge 104 comprises a top surface, a bottom surface, a front surface, and a rear surface. The shaving razor 100B may comprise of a primary working plane that comprises a primary blade group. The primary blade group may comprises one or more razor blade strips to shave broad and relatively flat areas of the face.

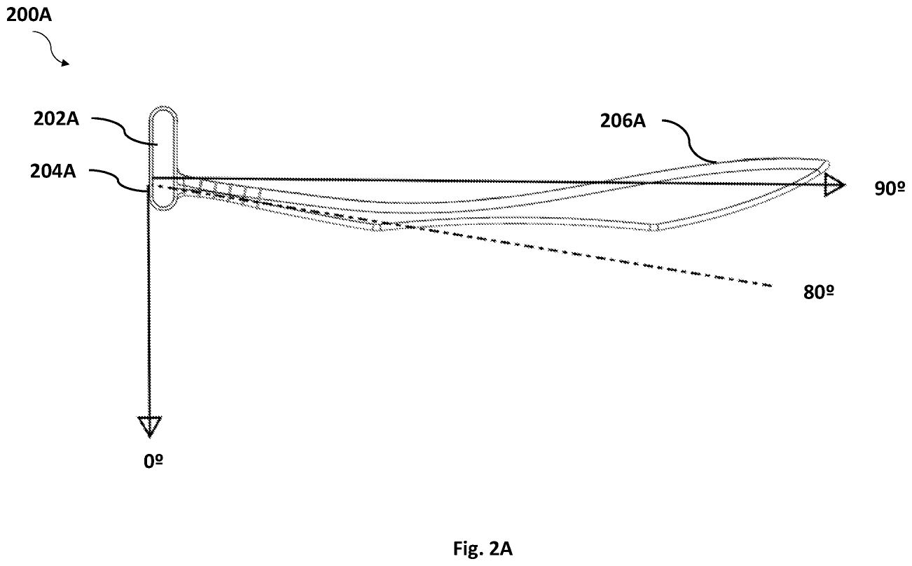

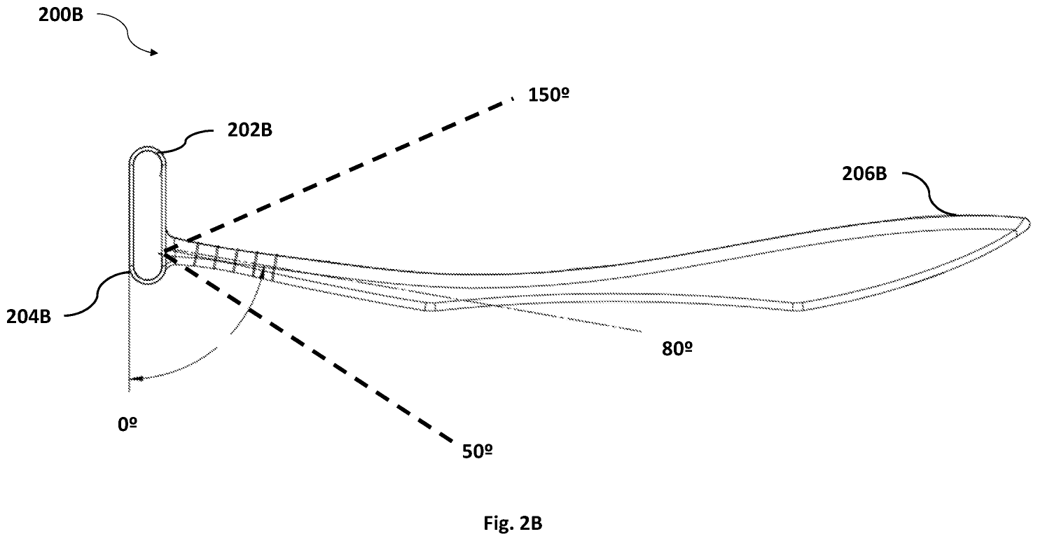

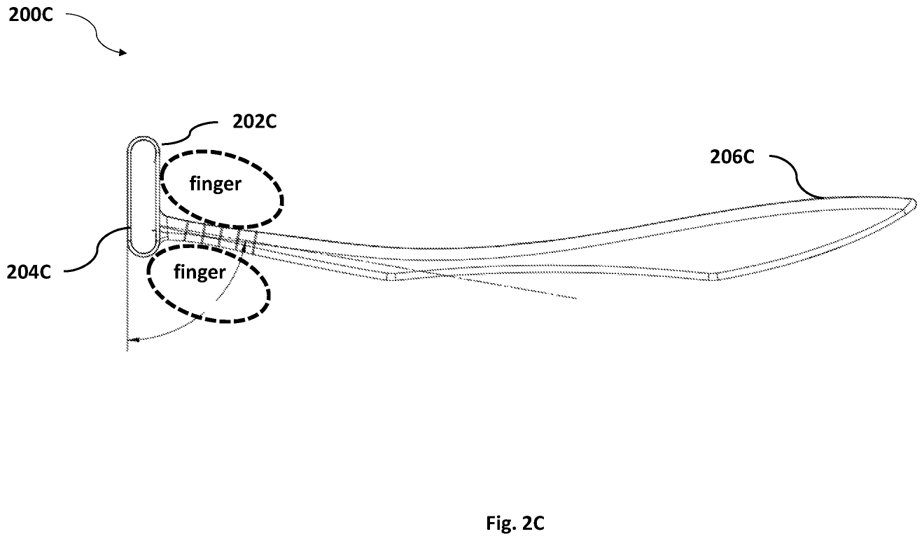

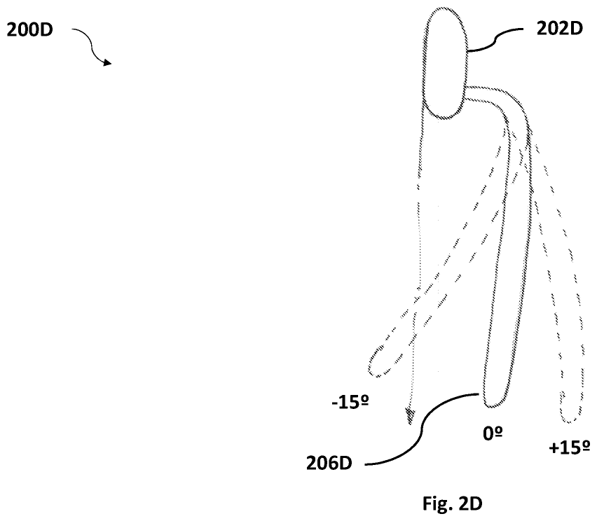

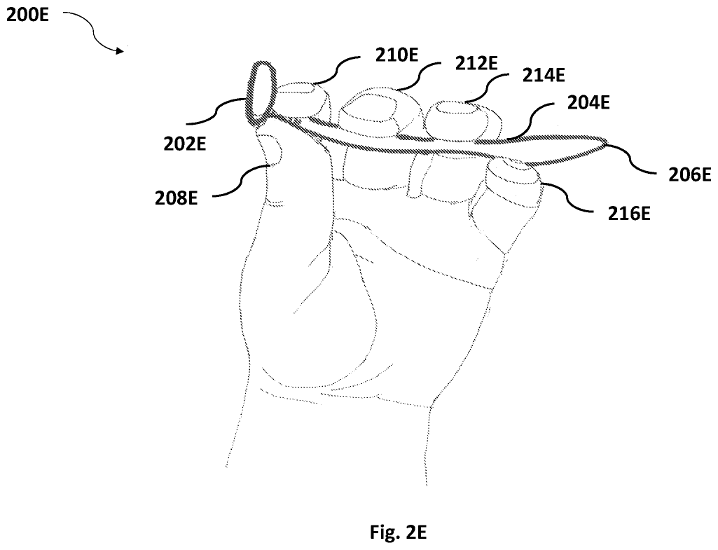

Referring to FIG. 2A illustrating an angular reference of head 202A with handle 206A of the shaving razor 200A at zero degrees, the front surface of the head 202A is placed against the anatomy 204A to be shaved. An anatomy contact surface is a front surface of the head 202A and used as the angular reference when the razor is viewed from its side. The direction of shaving defines the 0° angular reference. The distal length of the handle, preferably the 10-50 mm exposed curved length, lays in the +50° to +150° range, preferably in the 60° to 105° range, and more preferably 70°-95°. Proximal end of the razor to measure the angle is not the most accurate way to describe the head-handle angle of the present invention: the first 20-40 mm distal length extending from the rear surface of the head 202A is the portion that will be grabbed by the user.

Referring to FIG. 2B in an embodiment, there can be a possible scenario in which lower limit of the angular region is defined as a line at 50° passing through attachment point of the handle 206B (as viewed from the side) to the head 202B. Conversely, the upper limit of the angular region is defined as a line oriented at 150° passing through the attachment point of the handle 206B to the head 202B. In the preferred embodiment, the conventional curved length of the handle 206B, 10-30 mm with middle line lays in the 50°-150° range, preferably in the 60°-105°, more preferably 70°-95° and is designed to enable the user to grab the razor just behind the back surface of the razor head 202B.

Referring now to FIG. 2C of the present invention, illustrating an angular reference of head 202C with handle 206C of the shaving razor 200C, at an angle range of 70 degrees to 105 degrees, to shave the anatomy 204C. Cross sections of the user's thumb and index are represented to demonstrate the preferred handling, while optionally, an accurate rest for the middle finger can be provided. The area available to grab the handle 206C is located just at the back of the head 202C (or close by) extending from the back surface of the head 202C which is 0-50 mm, preferably 0-30 mm. The head-handle angle is within the 50°-150° range, preferably in the 60°-105°, range and more preferably in the 70°-95° range.

Referring to FIG. 2C illustrating an angular reference of head 202C with handle 206C of the shaving razor 200C, to shave the anatomy 204C. The handle 206C is connected to the back of the razor head 202C, optionally the design includes the pivot articulation with a spring back action to push the head toward the anatomy 204C.

Referring to FIG. 2D, typical handle designs of commercially available products start with a small offset (1-10 mm directed at approximately 60°-90° from the rear surface of the head 202D to then curve to drop toward the 0° direction. These commercially available razors are not designed to enable the user to grab the handle 206D just behind the razor head, instead it is conducive to grab the handle 206D right at the proximal location right past the curving down of the handle 206D.

FIG. 2E illustrates placement of fingers of the user to hold shaving razor 200E while shaving the anatomy (shaving area). An index finger 210E and a thumb finger 208E of the user is able to secure head 202E of the shaving razor 200E at a fixed position. A plurality of blades are mounted on front surface of the head 202E. A middle-finger 212E and a ring-finger 214E are positioned to rest in the middle portion of handle 204E. The pinky-finger 216E is positioned on bottom towards proximal tip 206E of the handle 204E. The razor handle 204E has a curved profile and is oriented at an angle relatively perpendicular to contact surface of the razor head 202E, thereby enabling the user to grab the razor handle 204E with fingers resting close to rear surface of the razor head 202E, touching the rear surface of the razor head 202E.

In some embodiments, a distal end of the handle 204E is connected to the back of the razor head 202E at a specific fixed angle relative to contact surface of the razor head 202E, and enabling the user to grab the razor handle 204E with gripping fingers resting close to rear surface of the razor head 202E. In other embodiments, the distal end fo the handle 204E is connected through a pivot or articulation.

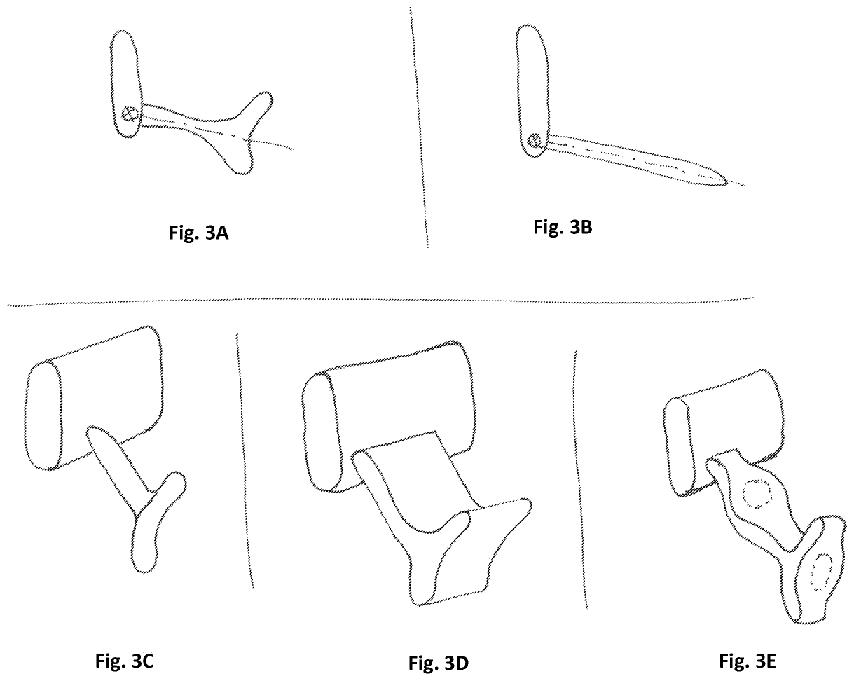

FIG. 3A-3E illustrates different types of handle designs with their respective profiles. Different variations of the handle profile and design are of flat cross section 3B, profiled cross section 3A, rod like 3C, extruded cross section 3D, round cross section or any other shape. The thickness of the handle increases with the distance from the back surface of the head. This does promote the “pulling” of the razor head towards the holding fingers.

In an alternate embodiment, the cross section of the handle is rod shaped or extruded X-section shaped.

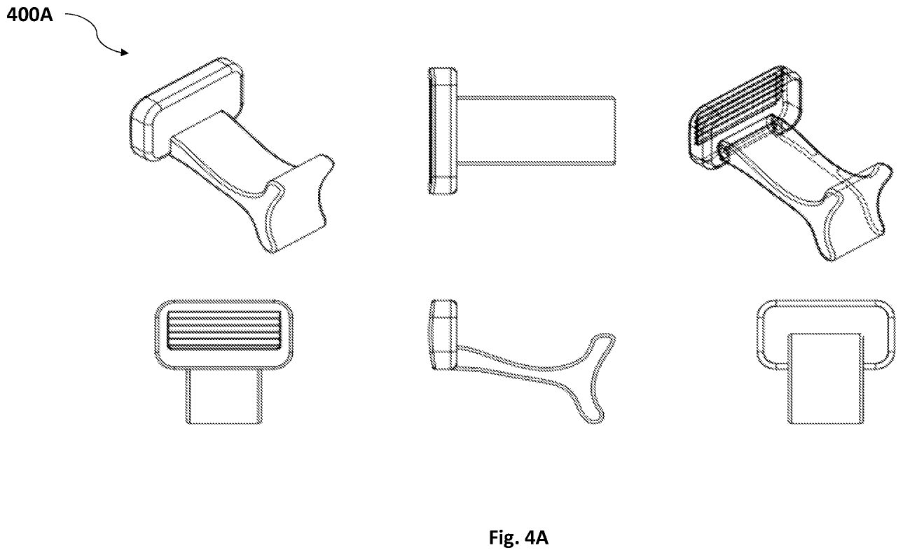

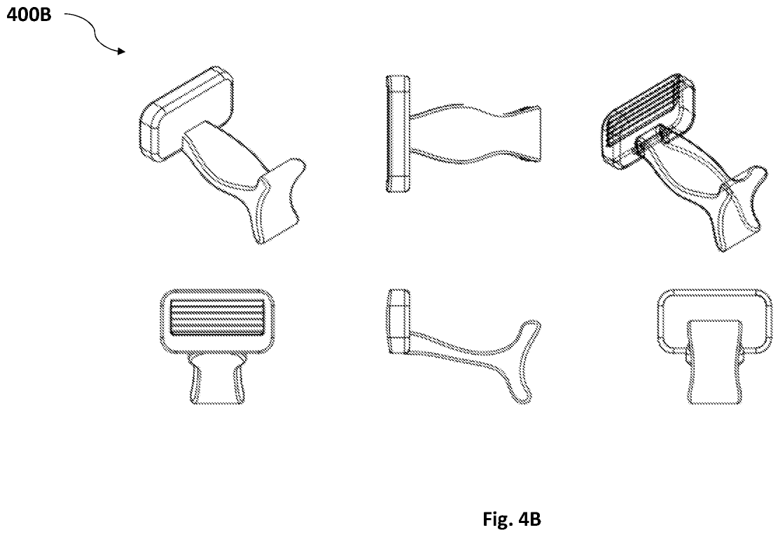

In one alternate embodiment, the profile of the handle 400A is in an extruded shape with reference to FIG. 4A of the present invention.

In another alternate embodiment, the profile of the handle 400B may be shaped with different structural forms such as shaped profile handle with reference to FIG. 4B of the present invention.

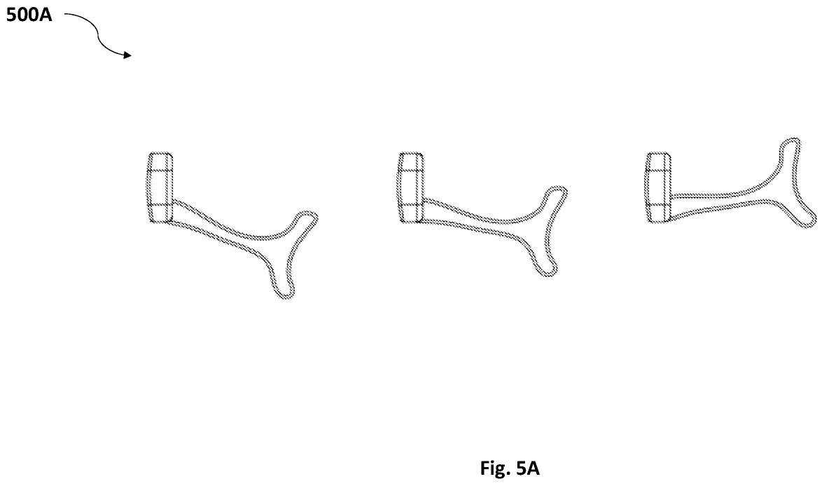

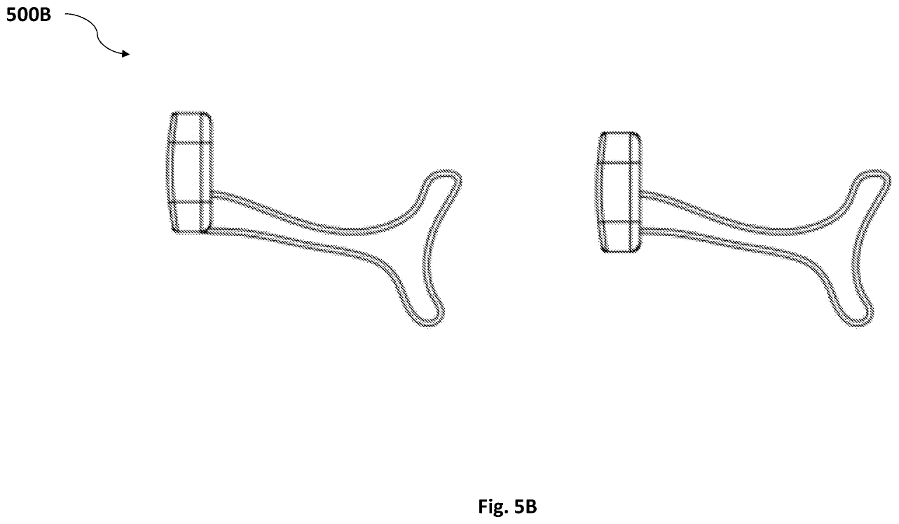

Referring to FIG. 5A illustrating various head-handle angle variations 500A, the head and the handle are arranged in with each other as the single design with and can be at different angular references ranging from 85-150 degrees.

Referring to FIG. 5B illustrating head-handle attachment point location variations 500B, there are different point locations where the attachment of head with the handle is at a certain angular reference range lying between 85-150 degrees.

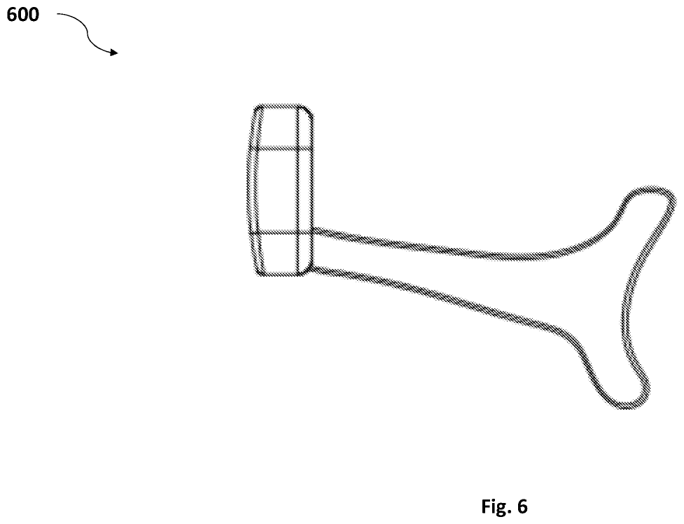

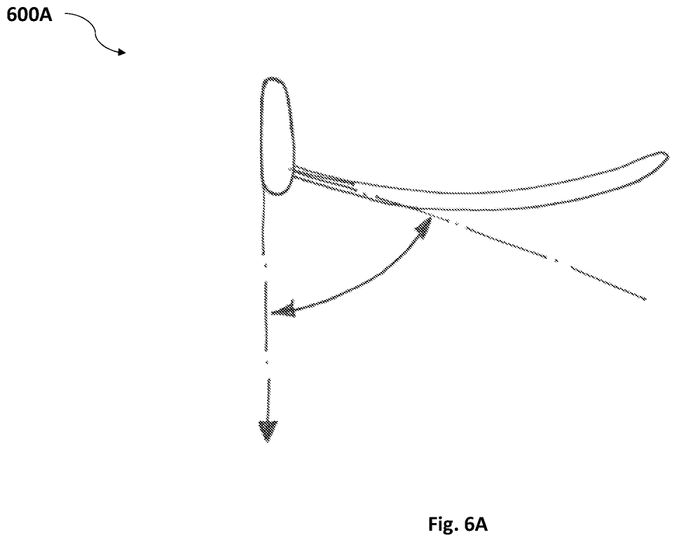

Referring to FIG. 6 illustrating an exemplary embodiment of the handle 600 with increasing thickness. The thickness of the handle 600 increases with the distance from the rear surface of the head. This facilitates the “pulling” of the razorhead towards the holding fingers.

Referring to FIG. 6A illustrating a conventional handle 600A with a preferred profile, the conventional handle 600A have handle curved with a center of curvature located towards the body (as per FIG. 6A). The conventional length handle is to be curved upward with a center of curvature away from the body. Unlike currently commercially-available razor, the handle is curved in an “upward” fashion going away from the 0° direction defined by the shaving direction. The handle is curved, and has a 70°-95° orientation with respect to the razor head front surface. It enables grabbing the handle just beyond the back surface of the razor head.

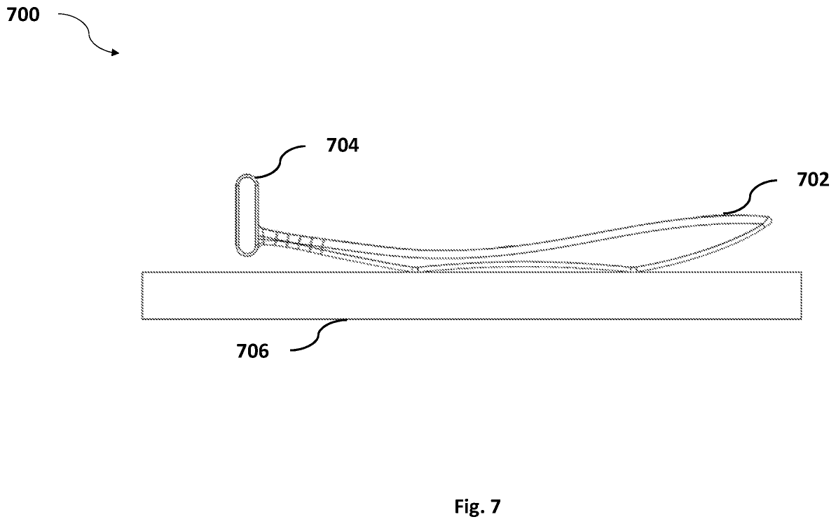

Referring to FIG. 7 illustrating a perspective view of the handle 702 of the shaving razor 700 lying on a flat surface 706. The handle 702 is designed in a way to lay in a stable fashion when resting on the flat surface 706, such as, countertop or table while maintaining the razor head 704 away from the table 706. This can be achieved in many different fashions: creating a flat profile on the bottom portion of the handle 702, or two small resting surface/features away from each other, or any other suitable means. The handle 702 is curved upward or away from body or angle between the front surface of the razor 700 and the distal portion of handle 702 is approximately 90°. Potential features for the razor blades also includes spring-mounted or flexible support mounted blades to allow movement of the blades within the razor head 704. The razor head 704 also comes under different variations in designs having different razor edges length, and also potentially including shaving strip/compound.

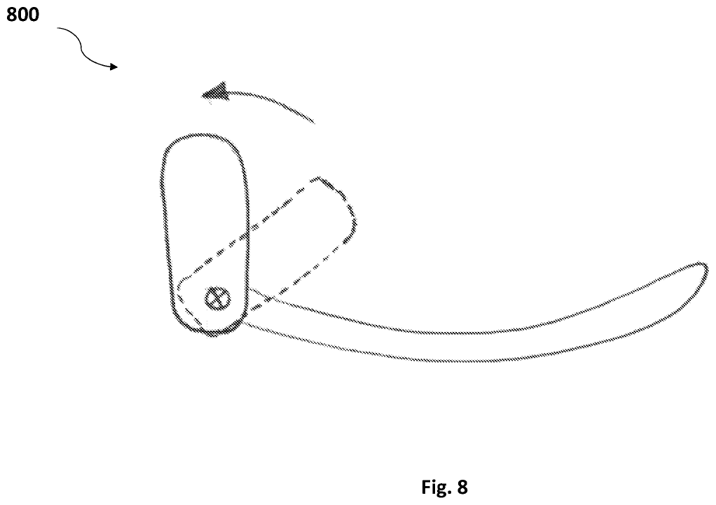

Referring to FIG. 8 illustrating head-handle pivot articulation and spring action 800 the zone of attachment between the handle and the head, along with the definition of a pivot articulation and its axis is shown. The pivot axis is preferably located toward the bottom surface of the razor head and its axis is parallel to the cutting edges of the blades.

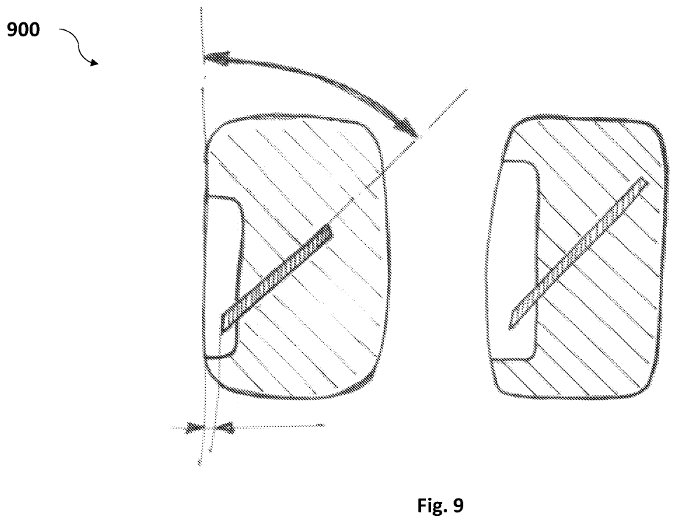

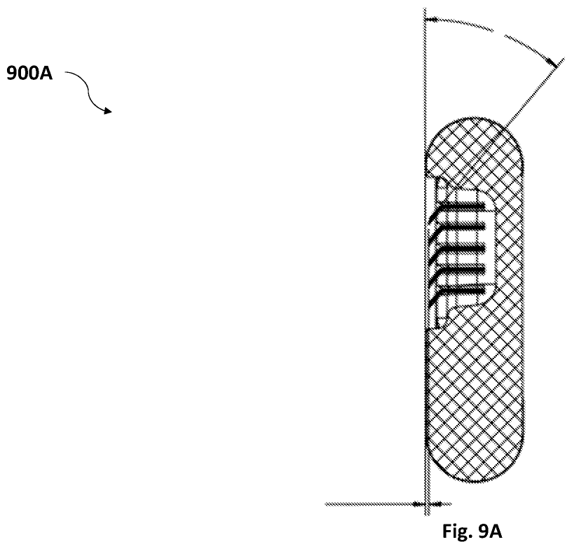

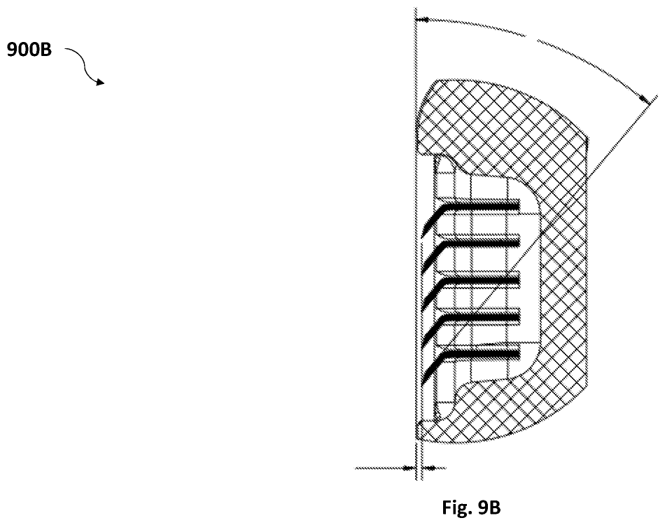

Referring to FIG. 9 illustrating razor head, blade, cutting angle and recess. A cutting angle is defined and the recess of cutting edge of the blade with respect to the head anatomy-contacting surface (defined by the head front surface).Potential features for the razor 900 shown in FIG. 9 also includes spring-mounted or flexible support mounted blades to allow movement of the blades within the razor head.

Referring to FIG. 9A illustrating angulation of blades with respect to the head of the shaving razor, the blades are at angle range of 45 degrees to 90 degrees. The blades of the shaving razor are mounted on a razor blade platform and a rear edge portion and a front edge portion facing opposite to each other. The razor blade may be made up of certain corrosion resistant materials such as steel, or blends of steel for example steel carbide which may be wider and thicker.

Referring to FIG. 9B illustrating angulation of blades with respect to the head of the shaving razor, the blades are at an angle range of 90 degrees to less than 45 degrees. The blades of the shaving razor are mounted in a manner that the user does not get shaving induced irritation or burns while carrying out shaving on the anatomy (the area which needs to be shaved). The blades as shown with respect to FIGS. 9A and 9B are not intended to limiting to the angulation of 90 degrees but can be also applicable to angles varying from 90 degrees (greater than 90 degrees as lesser than 90 degrees is illustrated).

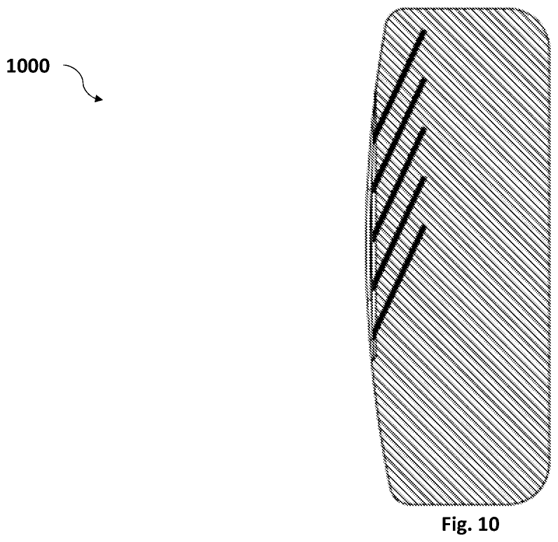

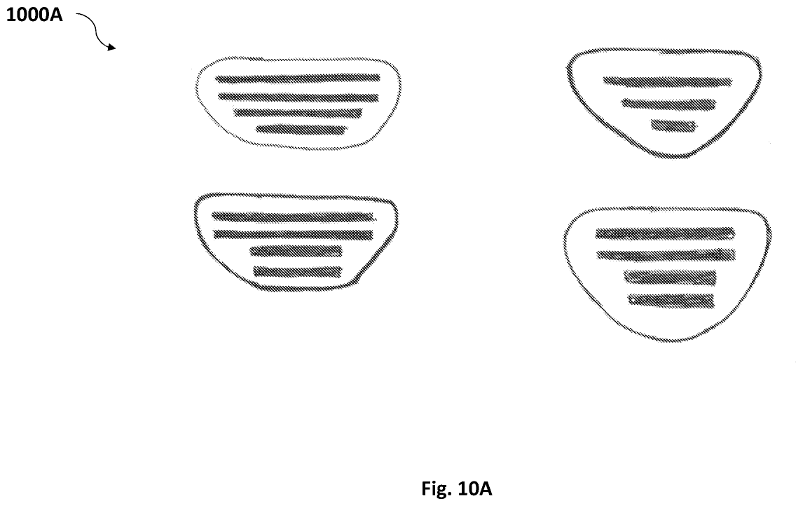

Referring to FIG. 10 illustrating razor head 1000 with finite number of blades, which have a curved anatomy contacting surface.

Referring to FIG. 10A illustrating variation in shape of razor heads 1000A having blade of different lengths. These different length are referred to razor edges with the different lengths. These razor edges may potentially include shaving strip/ compound.

It will also be understood that while the invention has been described with reference to embodiments illustrating the best method and apparatus currently known to the inventors, variations and modifications to the method and apparatus may be made without departing from the principles or concepts described herein.

The systems and methods disclosed herein can be implemented certain types of device configurations and methods are described herein for illustrative purposes, other types of device configurations and methods may be used.

The embodiments discussed herein are provided by way of example, and various modifications can be made to the embodiments described herein. Certain features that are described in this disclosure in the context of separate embodiments can also be implemented in combination in a single embodiment. Conversely, various features that are described in the context of a single embodiment can be implemented in multiple embodiments separately or in various suitable sub combinations. Also, features described in connection with one combination can be excised from that combination and can be combined with other features in various combinations and sub combinations.

Similarly, while operations are depicted in the drawings or described in a particular order, the operations can be performed in a different order than shown or described. Other operations not depicted can be incorporated before, after, or simultaneously with the operations shown or described. In some instances, certain operations described herein can be omitted or can be combined with other disclosed operations. In certain circumstances, parallel processing or multitasking can be used. Also, in some cases, the operations shown or discussed can be omitted or recombined to form various combinations and sub combinations.

The present invention has been described with reference to exemplary embodiments. However, it will be readily apparent to those skilled in the art that it is possible to embody the invention in specific forms other than those of the exemplary embodiments described above. This may be done without departing from the spirit of the invention. The described embodiments are merely illustrative and should not be considered restrictive in any way. The scope of the invention is given by the appended claims and their equivalents, rather than the preceding description, and all variations and equivalents which fall within the range of the claims are intended to be embraced therein. Although the invention has been shown and described with respect to certain embodiments, it is obvious that equivalent alterations and modifications will occur to others skilled in the art upon the reading and understanding of the specification. In particular, with regard to the various functions performed by the above-described components, the terms (including any reference to a “means”) used to describe such components are intended to correspond, unless otherwise indicated, to any component which performs the specified function of the described component (e.g., that is functionally equivalent) even though not structurally equivalent to the disclosed component which performs the functions in the herein exemplary embodiments of the invention. In addition, while a particular feature of the invention may have been disclosed with respect to only one embodiment, such feature may be combined with one or more other features of other embodiments as may be desired or advantageous for any given or particular application.

While embodiments of the present invention have been illustrated and described, it will be clear that the invention is not limited to these embodiments only. Numerous modifications, changes, variations, substitutions, and equivalents, will be apparent to those skilled in the art without departing from the spirit and scope of the invention, as described in the claims.

The foregoing description of embodiments is provided to enable any person skilled in the art to make and use the subject matter. Various modifications to these embodiments will be readily apparent to those skilled in the art, and the novel principles and subject matter disclosed herein may be applied to other embodiments without the use of the innovative faculty. The claimed subject matter outlined in the claims is not intended to be limited to the embodiments shown herein but is to be accorded to the widest scope consistent with the principles and novel features disclosed herein. It is contemplated that additional embodiments are within the spirit and true scope of the disclosed subject matter.

What is claimed is:

1. A shaving razor, comprising:
a razor head;
a plurality of razor blades mounted on front surface of the razor head; and
a razor handle having a curved profile, deviating away from razor body, wherein the razor handle is oriented at an angle relatively perpendicular to contact surface of the razor head, thereby enabling the user to grab the razor handle with fingers resting close to rear surface of the razor head, thereby touching the rear surface of the razor head.

2. The shaving razor as claimed in claim 1, further comprising orientation between the razor head and the razor handle within the range of 50 degrees to 150 degrees, preferably in the 60 degrees to 105 degrees, more preferably in the 70 degrees to 95 degrees.

3. The shaving razor as claimed in claim 1, wherein the razor handle is having conventional length of 80-180 mm preferably, thereby providing the user enough space to change grip and direction of the shaving razor.

4. The shaving razor as claimed in claim 1, wherein the razor handle is connected to the rear surface of the razor head.

5. The shaving razor as claimed in claim 1, wherein the contact surface of the razor head is flat or slightly convex with a large radius of curvature.

6. The shaving razor as claimed in claim 1, wherein the rear edge portion and the front edge portion of the razor head are slightly recessed from front face of the razor head.

7. The shaving razor as claimed in claim 1, wherein the razor head comprises a frame to protect the contact surface of the razor head from corners of the plurality of blades.

8. The shaving razor as claimed in claim 1, wherein the razor handle can be of flat cross section, profiled cross section, round cross section or any other suitable shape.

9. The shaving razor as claimed in claim 1, wherein the plurality of razor blades comprises spring mounted or support mounted blades which allows movement of the plurality of razor blades within the razor head.

10. The shaving razor as claimed in claim 1, further comprising a pivot axis of the razor handle positioned towards bottom surface of the razor head and parallel to cutting edges of the plurality of razor blades.

11. A shaving razor as claimed in claim 1, whereas the razor head can be integrated with razor head cartridge.

12. The shaving razor of claim 1, wherein orientation between distal end of the razor handle and the front surface of the razor head can be adjusted at any desired angle as required by the user.

13. The shaving razor of claim 1, whering the razor handle is configured to lay stable when resting on the flat support surface, while maintaining the razor head away from the support surface.

14. A shaving razor, comprising:
a razor head;
a razor head cartridge having a plurality of razor blades mounted on a razor blade platform and a rear edge portion and a front edge portion facing opposite to each other; and
a razor handle having a distal end connected to the back of the razor head at a fixed angle relative to contact surface of the razor head, and enabling the user to grab the razor handle with gripping fingers resting close to rear surface of the razor head.

15. The shaving razor as claimed in claim 14, wherein the razor handle distal portion is relatively straight (or slightly curved) for the first 10-30 mm length.

16. The shaving razor as claimed in claim 14, wherein the razor handle distal (gripping) portion is having length of 10-60 mm, thereby providing the user enough space to change grip and direction of the shaving razor, while the overall length of the razor handle lies in the 60-160 mm.

17. The shaving razor as claimed in claim 14, further comprising orientation between the razor head and the distal portion of the razor handle within the range of 50 degrees to 150 degrees, preferably in the 60 degrees to 105 degrees, more preferably in the 70 degrees to 95 degrees.

18. The shaving razor as claimed in claim 14, wherein the contact surface of the razor head is flat or slightly convex with a large radius of curvature.

19. The shaving razor as claimed in claim 14, wherein the rear edge portion and the front edge portion of the razor head are slightly recessed from front face of the razor head.

20. The shaving razor as claimed in claim 14, wherein the razor head comprises a frame to protect the contact surface of the razor head from corners of the plurality of blades.

21. The shaving razor as claimed in claim 14, wherein the razor handle can be of flat cross section, profiled cross section, round cross section or any other suitable shape.

22. The shaving razor as claimed in claim 14, wherein the plurality of razor blades comprises spring mounted or support mounted blades which allow movement of the plurality of razor blades within the razor head.

23. A shaving razor as claimed in claim 14, whereas the razor head can be integrated with razor head cartridge.

24. The shaving razor as claimed in claim 14, wherein orientation between the distal end of the razor handle and the front surface of the razor head can be adjusted at any desired angle as required by the user.

25. The shaving razor of claim 14, whering the razor handle is configured to lay stable when resting on the flat support surface, while maintaining the razor head away from the support surface.
